(19) United States
(12) Patent Application Publication
(10) Pub. No.: US 2018/0126191 A1
(43) Pub. Date: May 10, 2018

(54) METHODS FOR REDUCING INFLAMMATION WITH SURFACE ACOUSTIC WAVES

(71) Applicant: The Trustees Of Columbia University In The City Of New York, New York, NY (US)

(72) Inventors: John D. Loike, New York, NY (US); Vernice Jackson-Lewis, New York, NY (US); Serge Przedborski, New York, NY (US)

(21) Appl. No.: 15/807,974

(22) Filed: Nov. 9, 2017

Related U.S. Application Data
(60) Provisional application No. 62/419,562, filed on Nov. 9, 2016.

(51) Int. Cl. A61N 7/00; C07K 16/18; A61K 39/395; A61M 37/00

(52) U.S. Cl. CPC A61N 7/00; C07K 16/18; A61M 2037/0007; A61M 37/0092; A61K 39/3955

(57) ABSTRACT

Disclosed herein is a method of reducing inflammation in a patient with a chronic condition, such as Parkinson's disease or Alzheimer's disease. The method includes applying surface acoustic waves to the patient, such that the surface acoustic waves promote receptor mediated uptake of a target protein. For Parkinson's disease, the application of SAW promoted Fc-receptor mediated uptake of α-synuclein to reduce inflammation in the patient and alleviate symptoms related to Parkinson's disease.

### CROSS REFERENCE TO RELATED APPLICATIONS

This application claims the benefit of application No. 62/419,562 filed on Nov. 9, 2016 which is hereby incorporated herein by reference in its entirety.

### STATEMENT REGARDING FEDERALLY SPONSORED RESEARCH OR DEVELOPMENT

This invention was made with government support under W81XWH-08-1-0465 awarded by Army/MRMC and NS099862 awarded by the National Institutes of Health. The government has certain rights in the invention.

### BACKGROUND

Ultrasound is a form of mechanical energy that is transmitted into biologic tissues as an acoustic pressure wave at specified frequencies. At high frequencies ultrasound can cause significant cell and tissue damage. Surface acoustic waves (SAW) therapy uses a scattered beam whose energy is almost totally absorbed at the surface of the tissue, minimizing tissue damage. However, because the mechanism of action of SAW is unknown, applications of SAW for therapeutic purposes have been limited.

Parkinson's disease (PD) is the second most common, late onset, irreversible neurodegenerative disorder. The neuropathological change primarily responsible for the clinical decline in PD is a progressive loss of the dopamine (DA)-containing neurons in the substantia nigra pars compacta (SNpc). There is also a loss of their connecting terminals in the corpus striatum as well as the buildup of intraneuronal inclusions, called Lewy bodies, which contain alpha-synuclein (α-syn), neuromelanin, and ubiquitin.

### SUMMARY

This disclosure provides a method for promoting microglial cell ingestion and clearance of toxic proteins associated with Parkinson's disease and Alzheimer's disease and reducing inflammation through the application of surface acoustic waves. More particularly, the disclosure provides a way to treat chronic conditions such as Parkinson's disease or Alzheimer's disease (AD) by reducing inflammation and promote the clearance of toxic proteins in the patient at an area of interest. SAW can promote the receptor-mediated uptake of a target protein. For Parkinson's disease, the application of SAW promotes the Fc-receptor-mediated uptake of α-syn. Because the extracellular deposition of α-syn is likely a critical inflammatory component of PD, the promotion of α-syn uptake will lead to a reduction in inflammation in the patient and possibly alleviate the symptoms and pathology related to Parkinson's disease. The promotion of uptake of α-syn, beta amyloid, or other target neurologically toxic proteins by SAW can also be further applied to the reduction of inflammation in other chronic conditions, such as Alzheimer's disease. The reduction of inflammation by SAW can be quantified by the reduction in the secretion of TNF-α or IL-6 in the area of interest.

SAW can also be used to open the blood brain barrier (BBB) to allow the entry of intravenous (IV) or intraperitoneal (i.p.) administered antibodies from the vasculature into the central nervous system (CNS). The administered antibodies can be antibodies to the neurologically toxic proteins, such as α-syn or beta amyloid antibodies. The blood brain barrier (BBB) is known to restrict the entry of large molecules, such as antibodies, from the vascular system into the CNS. Thus, SAW would open the BBB and allow the antibodies to form immune complexes in the pathological regions of the brain as well as promote the Fc-mediated uptake of synuclein or beta amyloid deposits. In addition to promoting the entry of antibodies in the CNS, SAW can be used to promote the entry of antibodies into the CNS that may improve treatments to PD, AD, and CNS targeted cancers.

A plurality of particles coated with antibodies can also be administered with the application of SAW, such that SAW promotes uptake of the particles. The particles can be further used as a therapeutic against the chronic condition. The particles can include anti-α-synuclein antibodies, beta amyloid antibodies, other therapeutic antibodies, or therapeutic agents for the chronic condition. The α-synuclein or beta amyloid on the surface of the particles may be used to target the particles to the area of interest, as the application of SAW will promote the uptake of the particles, along with any therapeutic on or incorporated in the particles. Alternatively, other therapeutic antibodies may be administered with the application of SAW without being incorporated into a particle.

Additional embodiments and features are set forth in part in the description that follows, and will become apparent to those skilled in the art upon examination of the specification or may be learned by the practice of the disclosed subject matter. A further understanding of the nature and advantages of the disclosure may be realized by reference to the remaining portions of the specification and the drawings, which form a part of this disclosure.

### BRIEF DESCRIPTION OF THE DRAWINGS

The application file contains at least one drawing executed in color. Copies of this patent application publication with color drawing(s) will be provided by the Office upon request and payment of the necessary fee.

The description will be more fully understood with reference to the following figures and data graphs, which are presented as various embodiments of the disclosure and should not be construed as a complete recitation of the scope of the disclosure. It is noted that, for purposes of illustrative clarity, certain elements in various drawings may not be drawn to scale. Understanding that these drawings depict only exemplary embodiments of the disclosure and are not therefore to be considered to be limiting of its scope, the principles herein are described and explained with additional specificity and detail through the use of the accompanying drawings in which:

FIG. 1 illustrates the binding and uptake to protein-coated fluorescent microspheres by cells on which the various receptors have been down-regulated according to some embodiments of the disclosed subject matter. This method is used to assess the capacity of microglial cells or other cells of ingesting specific proteins or antibody opsonized proteins.

FIG. 2 is a graph of the results of an adherence assay showing that α-syn promotes adherence of microglia according to some embodiments of the disclosed subject matter. Error bars=SEM.

FIG. 3 is a graph of the results of an adherence assay showing that anti-CD11b and anti-SR-B2 antibodies reduce the adherence of BV2 and N9 microglia to α-syn according to some embodiments of the disclosed subject matter. Error bars=SEM.

FIG. 4 is a graph showing the results of a down-modulation assay to identify CD11b and SR-B2 as important receptors mediating α-syn ingestion according to some embodiments of the disclosed subject matter. Error bars=SEM.

FIG. 5 is a graph showing the effects of anti-CD11b and anti-SR-B2 on the ingestion of α-syn and fibrillar α-syn (PFF) by N9 according to some embodiments of the disclosed subject matter.

FIG. 6 is a graph of the results of a down-modulation assay showing that anti-α-syn promotes α-syn uptake independent of CD11b and SR-B2 receptors according to some embodiments of the disclosed subject matter. Error bars=SEM.

FIG. 7 is a graph showing the effects of ant-CD11b and anti-SR-B2 on the ingestion of monomeric vs oligomeric proteins according to some embodiments of the disclosed subject matter.

FIG. 8A is a confocal microscopy image of N9 cells ingesting monomeric α-syn microspheres merged image from the stack of 14 optical slices according to some embodiments of the disclosed subject matter.

FIG. 8B is a single optical slice of a confocal microscopy image of N9 cells ingesting monomeric α-syn according to some embodiments of the disclosed subject matter.

FIG. 8C is a single optical slice of a confocal microscopy image of N9 cells ingesting monomeric α-syn according to some embodiments of the disclosed subject matter.

FIG. 8D is a merged confocal microscopy image of N9 cells with microspheres attached to the plasma membrane according to some embodiments of the disclosed subject matter.

FIG. 9 is a graph showing the clearance of matrix-bound monomeric or PFF α-syn by N9 cells according to some embodiments of the disclosed subject matter. *statistically significant at p<0.01; n=4; Error bars=SEM.

FIG. 10 is a graph showing the effect of SAW on α-syn uptake according to some embodiments of the disclosed subject matter.

FIG. 11 is a graph showing the effect of SAW on TNF and IL-6 production (biomarkers for inflammation) according to some embodiments of the disclosed subject matter.

FIG. 12 is a graph showing the effect of SAW on IL-6 production with various substrates and coated spheres according to some embodiments of the disclosed subject matter.

FIG. 13 is a graph showing the effects of SAW on N9 ingestion of protein-coated spheres according to some embodiments of the disclosed subject matter.

### DETAILED DESCRIPTION

Various embodiments of the disclosure are discussed in detail below. While specific implementations are discussed, it should be understood that this is done for illustration purposes only. A person skilled in the relevant art will recognize that other components and configurations may be used without parting from the spirit and scope of the disclosure. Thus, the following description and drawings are illustrative and are not to be construed as limiting. Numerous specific details are described to provide a thorough understanding of the disclosure. However, in certain instances, well-known or conventional details are not described in order to avoid obscuring the description. References to one or an embodiment in the present disclosure can be references to the same embodiment or any embodiment; and, such references mean at least one of the embodiments.

Reference to “one embodiment” or “an embodiment” means that a particular feature, structure, or characteristic described in connection with the embodiment is included in at least one embodiment of the disclosure. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others.

The terms used in this specification generally have their ordinary meanings in the art, within the context of the disclosure, and in the specific context where each term is used. Alternative language and synonyms may be used for any one or more of the terms discussed herein, and no special significance should be placed upon whether or not a term is elaborated or discussed herein. In some cases, synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any example term. Likewise, the disclosure is not limited to various embodiments given in this specification.

Provided herein is a method of reducing inflammation for treatment of a chronic medical condition. The method includes applying surface acoustic waves (SAW) to an area of interest in a patient with the chronic condition. The surface acoustic waves then reduce inflammation mediated by promoting receptor-mediated uptake of a target neurologically toxic protein. In an embodiment, the target proteins include monomeric alpha-synuclein (α-syn), mutated alpha synuclein, and fibrillar forms of the synuclein species. In another embodiment, target proteins include monomeric and oligomeric forms of beta amyloid. SAW inhibits the production of biomarkers of inflammation as microglial cells ingest the target protein. Therefore, a reduction in the secretion of inflammation biomarker can indicate that inflammation has been reduced in the area of interest as a result of the ingestion of the target neurologically toxic protein. The application of SAW can be used as a therapy for or treatment of various chronic conditions to be described below.

In one embodiment, the chronic condition is Parkinson's disease (PD). PD is characterized by the degeneration of specific subsets of neurons due to a mechanism that remains enigmatic. In rare instances, PD is caused by mutations or multiplication of the gene encoding for a small synaptic protein called alpha-synuclein (α-syn). α-syn is a soluble, 140 amino acid, predominantly presynaptic protein that is well-conserved among vertebrates. Although α-syn is an intracellular native protein, it can be deposited into the extracellular space due to, at least two, non-mutually exclusive mechanisms, namely atypical secretion and leakage from healthy and damaged neurons, respectively. It can be released by stressed or dying neurons to activate microglia, and trigger PD. Once in the extracellular environment, α-syn can be subjected to fertilization, oligomerization, and/or modification (nitration, phosphorylation) that can trigger a microglial-derived inflammatory response and a “prion-like” cell to cell spreading. It is found mainly in the hippocampus, frontal cortex, and striatum. Five missense mutations (A53T, A30P, E46K, H500, G51D) as well as fibrillary α-syn have been linked to genetic forms of PD. In addition, the release of α-syn by dying neurons can lead to extracellular α-syn fibrillation that can be detected in plasma and CSF of PD patients.

Aside from the dramatic loss of dopaminergic neurons, the SNpc is also the site of a marked gliosis in both human PD and experimental animal models of PD. This glial response may be a critical inflammatory component in PD pathogenesis as a result of the extracellular deposition of α-syn. Mounting evidence indicates that a fraction of mutated or overexpressed synuclein accumulates extracellularly, hence raising the possibility that a synuclein-induced neuroinflammatory response may contribute to the neurodegeneration seen in PD.

Two N9 microglial receptors have been identified, the β-integrin receptor CD11b/CD18 and scavenger receptor SR-B2, that mediate various interactions between microglial cells and matrix-bound monomeric (native) and oligomeric (fibrillary) α-syn or monomeric beta amyloid or oligomeric beta amyloid. These receptors play a critical role in the uptake and clearance of matrix-bound monomeric (native) and oligomeric (fibril) α-syn and beta amyloid. SR-B2, and the β-integrin receptor, CD11b/CD18, mediate cell adhesion to α-syn-containing matrices. Both CD11b integrins and SR-B2 scavenger receptors mediate N9 ingestion of native α-syn and monomeric beta amyloid. CD11b mediates N9 ingestion of fibrillar α-syn and oligomeric beta amyloid.

The roles of SR-B2 and CD11b/CD18 were examined in the uptake and clearance of both monomeric and oligomeric forms of mutated and wild-type synucleins and show how selected cytokines can either enhance or inhibit these processes. It was found that microglia clearance of matrix bound α-syn species (e.g. preformed fibrils [PFFs]) that has been linked to PD less efficiently than the species (e.g. native monomeric α-syn) which has not been linked to PD. Moreover, opsonization of PFFs enhances N9 ingestion of α-syn as well as the production of key proinflammatory factors such as TNFα and IL-6. Therefore, a mechanism that reduces the build-up of synuclein species in the CNS, without triggering a robust neuroinflammatory response, can be used as an effective therapy for PD.

Surface Acoustic Waves (SAW) are low-energy elastic ultrasound waves that are non-thermal. The vibration energy can be transmitted directly to indwelling medical devices or tissue in an integrated unit. SAW has been studied as a means to reduce infections that can occur in individuals who receive a prosthetic device. A proposed mechanism for SAW efficacy is its ability to enhance white blood cell invasion of the bacterial plaque on an implanted device and promote bacterial killing. In addition, SAW has also been shown to promote the killing of melanoma cells in vitro. Without being limited to a particular theory, SAW can serve as a trigger to activate a variety of leukocyte mechanoreceptors.

Integrins such as CD18 may function as mechanoreceptors. As such, CD18/CD11b integrins or SR-B2 scavenger receptors are sensitive to low energy ultrasound, such as SAW, and SAW can activate these receptors. Therefore, SAW can be used to affect target protein uptake and clearance, target protein mediated inflammation, and opening the BBB. In various embodiments, the target protein is α-syn or beta amyloid. For example, when SAW is applied to N9 cells, the ingestion of opsonized PFFs is enhanced, and the production of proinflammatory factors, such as IL-6, are dramatically reduced. Therefore, SAW, by promoting the ingestion of extracellular disease-related proteins like α-syn, reduces the microglial-derived inflammatory response, and in turn can reduce the cell-to-cell transmission of these toxic proteins, and the ensuing neurodegeneration. In an embodiment, SAW promotes Fc-receptor-mediated uptake of α-syn. SAW may also promote the ingestion of complement opsonized proteins via the CD11b receptor. Because of the promotion of uptake of α-syn, SAW reduces inflammation triggered by the interaction of α-syn with microglial cells.

Excess deposition of native α-syn or its modified (mutated or fibrillary forms) may not be cleared effectively by microglia in PD and other neurodegenerative diseases. Therefore, in an example, specific activation of CD11b/CD18, via SAW, can enhance microglial clearance of the various forms of α-syn and beta amyloid. Both CD11b integrins and SR-B2 scavenger receptors mediate N9 ingestion of native α-syn and monomeric beta amyloid; CD11b mediates N9 ingestion of fibrillar α-syn and oligomeric beta amyloid. Administration of SAW promotes the uptake of α-syn by microglia. In various embodiments, the administration of SAW can increase the uptake of α-syn or beta amyloid by microglia as much as two-fold.

What was unexpected and significant is that SAW also promotes Fc-mediated uptake of particles coated with α-syn and with an anti-α-syn antibody. Thus, by effective targeting of α-syn antibodies into the pathological regions of PD in the brain, SAW can be used to promote an antibody therapy against α-syn to attenuate PD or other chronic synucleinopathies. Thus, effective targeting of anti-syn antibodies into the pathological regions of PD in the brain may attenuate PD or other chronic synucleinopathies. In mouse models, this therapy seems to work with the caveat that microglia cells in the brains are taking up antibody opsonized to extracellular α-syn deposits more efficiently.

In various embodiments, SAW can promote the ingestion of particles coated with target proteins, antibodies to the target proteins, or combinations thereof. Non-limiting examples of the target proteins and antibodies include α-syn, beta amyloid, anti-α-syn, anti-beta amyloid, or combinations thereof. Antibodies directed against α-syn promote the uptake of α-syn-coated and fibrillar α-syn-coated particles or microspheres (for example, fl-spheres). Antibodies directed against monomeric beta amyloid promote the uptake of monomeric beta amyloid-coated and oligomeric beta amyloid-coated particles or microspheres (for example, fl-spheres). The α-syn on the surface of the particles is an effective method to assess the efficacy of α-syn ingestion to the area of interest, as the application of SAW will promote the uptake of the particles.

An important parameter in antibody therapy is the regulation of excess inflammation. It is clear that inflammation plays a role in PD as well as in many other diseases. Therefore, administration of SAW can dramatically decrease the release of TNF-α and IL-6 biomarkers by microglial cells that secrete these two important biomarkers of inflammation. Thus, in an embodiment, SAW promotes the uptake of α-syn and reduces synuclein-triggered inflammation.

In other embodiments, SAW can be applied to a patient or animal to reduce inflammation in other diseases with chronic inflammation, including but not limited to PD, Alzheimer's disease, diabetes, food allergies, transplantation rejection, cancer, and erectile dysfunction. In one embodiment, the application of SAW could be used to break up beta-amyloid plaques in Alzheimer's disease.

In alternative embodiments, focused ultrasound can be used to open the BBB to facilitate entry of anti-α-syn into the brain for further therapy and treatment of PD or AD. For example, SAW can be administered to the head of a patient or animal with Parkinson's disease or Alzheimer's disease to open the BBB. In an embodiment, anti-α-synuclein antibody or anti-beta-amyloid antibody can be simultaneously injected with SAW to allow for more antibodies cross the blood brain barrier to target the brain in the presence of SAW as a therapy for PD or Alzheimer's disease. In addition to promoting the entry of anti-synuclein of beta amyloid antibodies in the CNS, SAW could be used to promote the entry of antibodies into the CNS that may improve treatments to Alzheimer's disease and CNS targeted cancers. It is known that antibodies directed against breast cancer cells found in the periphery are effective means of eradication of the cancer. In contrast, when oncogenic breast cancer cells metastasized into CNS, these antibodies are non-effective because the failure to deliver the antibodies across the blood brain barrier into the CNS.

In another embodiment, SAW can be administered to the head of a patient or animal in conjunction with therapeutic antibodies that attenuate other aspects of the chronic condition. For example, SAW can be administered with therapeutic antibodies which attenuate behavioral deficits in either PD or Alzheimer's disease. In the presence of SAW, fewer antibodies will be required or the antibodies will have a greater impact on resolving the behavioral deficits.

In an embodiment, a wearable ultrasound device or targeted SAW device can be applied to the surface of the area of interest of the patient to deliver SAW. This device can be battery powered and supply SAW over a period of time. In various embodiments, SAW can be applied to the area of interest over about 15 minutes, about 30 minutes, about 1 hour, about 2 hours, about 3 hours, about 4 hours, about 5 hours, about 6 hours, about 7 hours, or about 8 hours. The time period of SAW application can be without interruption or can be further broken up into periods separated by a rest period without SAW application. The rest periods can range from about 10 minutes to about 30 minutes. The period of time SAW is applied to the area of interest can be repeated for about 1 day, about 2 days, about 3 days, about 4 days, about 5 days, about 6 days, about 1 week, or about 2 weeks.

In another embodiment, the power of SAW applied to the area of interest can range from about 0.05 W to about 1 W, from about 0.1 W to about 0.3 W, from about 0.2 W to about 0.4 W, from about 0.3 W to about 0.5 W, from about 0.4 W to about 0.6 W, from about 0.5 W to about 0.7 W, from about 0.6 W to about 0.8 W, from about 0.7 W to about 0.9 W, and from about 0.8 W to about 1 W. In one embodiment, the power of SAW is about 0.2 W or about 0.5 W. In other embodiments, the acoustic intensity of the device administering SAW can range from about 0.07 W/cm² to about 0.4 W/cm², from about 0.1 W/cm² to about 0.3 W/cm², and from about 0.2 W/cm² to about 0.4 W/cm². The frequency of the SAW applied to the target area can range from about 80 kHz to about 110 kHz. In one embodiment, the frequency is about 90 kHz.

### EXAMPLES

### Example 1: α-Synuclein Activates N9 Microglia

Activation of N9 fetal murine microglia cells was measured using TNF-α production, adherence, and chemotaxis. Adhesion was measured by adding 50,000 N9 cells to 96 well-plates coated with either 3 μg/well of laminin or 3 μg/well of laminin and α-syn. Chemotaxis was measured in cell culture inserts coated with either 3 μg/insert of laminin or 3 μg/insert of laminin and α-syn. Cytokine production of TNF-α and ROS production was measured in the media after a 48 hour incubation of N9 cells adherent to 3 μg/well laminin or 3 μg/well of laminin and α-syn. Table 1 presents the comparisons between laminin or laminin and α-syn cultures on N9 adhesion, chemotaxis, cytokine production, and ROS production.

TABLE 1

| Microglial Cell | | |
|---|---|---|
| Interactions | Laminin | Synuclein |
| Adhesion | 50% of cells added | 70-80% of cells added |
| Chemotaxis MCP-1 | 10% | 3-4% |
| Cytokine production | 100 pg/10⁵ cells | 500 pg/10⁵ cells |
| TNF-α | | |
| ROS Production | 2 fold increase | 2-3 fold increase |
| LPS/no LPS (90 min) | | |

### Example 2: α-Syn Promotes Adherence of Microglia

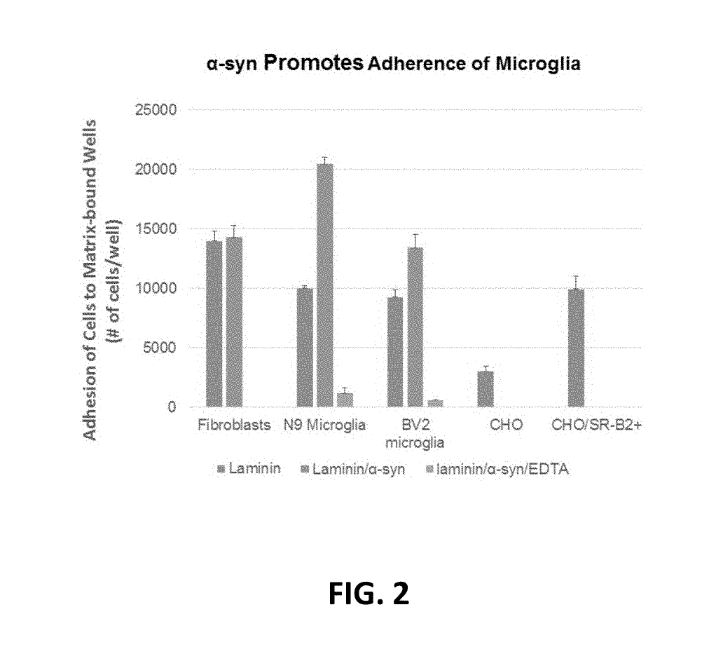

As seen in FIG. 2, an adherence assay was performed in which 50,000 cells were added to each well of a 96 well-plate for 2 hours in wells that were pre-coated with laminin or laminin/α-syn (3 μg/well). Non-adherent cells were washed away and the number of remaining cells was measured using a CyQuant assay. In the indicated wells, 10 mM EDTA was added to the cells. CHO/SR-B2⁺ are genetically modified to express the scavenger receptor SR-B2. Error bars=SEM.

### Example 3: Anti-CD11b and Anti-SR-B2 Antibodies Reduce the Adherence of BV2 and N9 Microglia to α-Syn

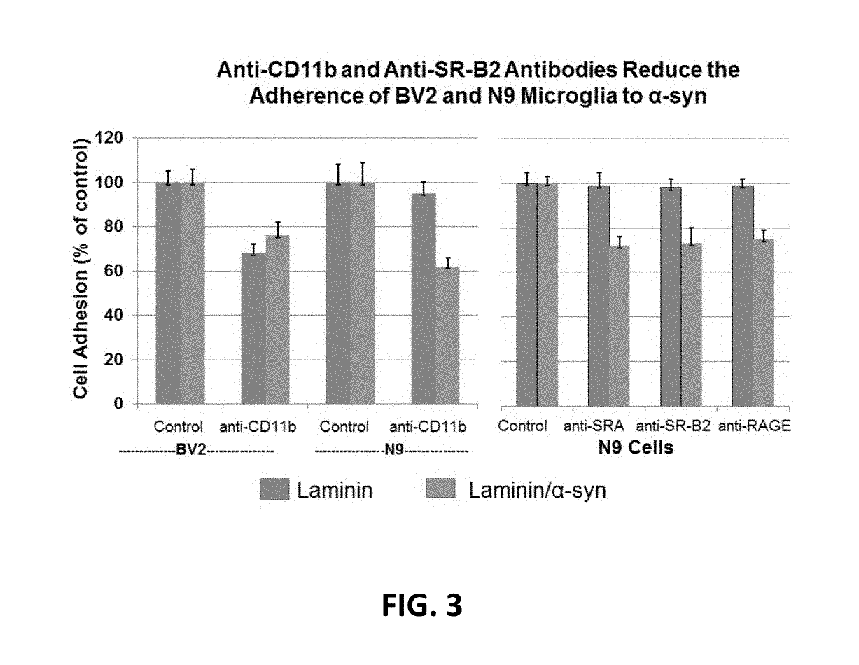

As seen in FIG. 3, an adherence assay was performed in which 50,000 cells (N9 or BV2) were added in the presence or absence of 10 μg/ml of the indicated antibodies to each well of a 96-well plate for 2 hours in which wells were pre-coated with either laminin or laminin and α-syn as described in Example 2. Non-adherent cells were washed away and the number of remaining adherent cells was assessed using a CyQuant assay. Error bars=SEM.

### Example 4: Identifying CD11b and SR-B2 by a Down-Modulation Assay

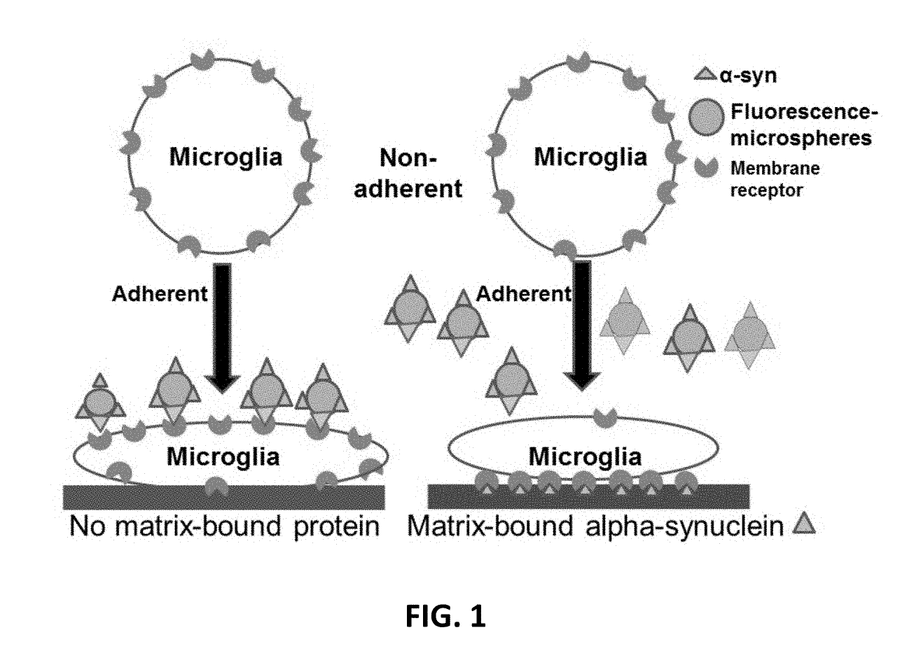

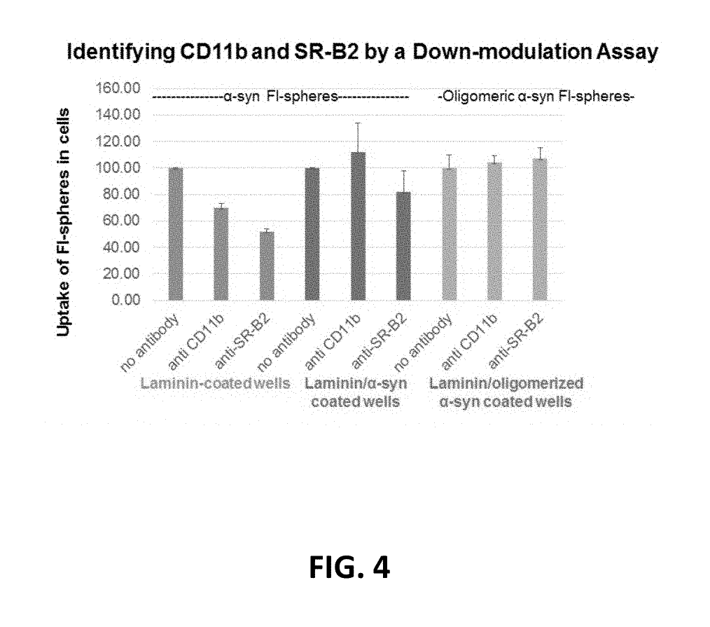

As illustrated in FIG. 1, an assay was performed in which phagocytosis of protein-coated fluorescent microspheres (Fl-spheres) was observed. As seen in FIG. 4, the binding and uptake of protein-coated Fl-spheres by cells on which the various receptors have been down-modulated was observed. 100,000 N9 cells were added to wells pre-coated with laminin, laminin and α-syn, or laminin and oligomeric α-syn for 24 hours. Non-adherent cells were removed and either α-syn or oligomeric α-syn coated Fl-spheres (100 beads/cell) were added to the wells for 60 minutes. The wells were then washed 3× in PBS and fluorescence was measured using a cell plate reader. All values are normalized for the number of cells adherent to each well as measured using the CyQuant assay.

### Example 5: Effects of Anti-CD11b and Anti-SR-B2 on the Ingestion of α-Syn and Fibrillar α-Syn (PFF) by N9

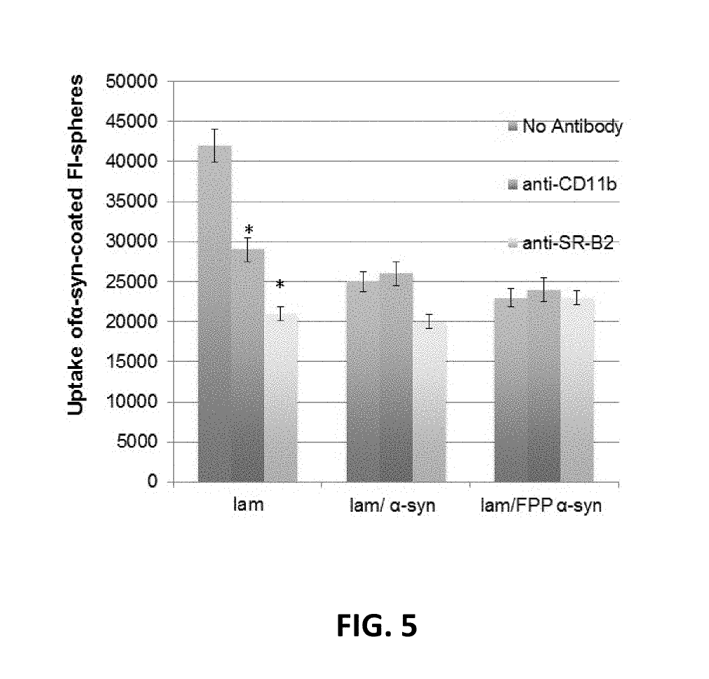

The same assay as Example 4 was performed with several modifications. As seen in FIG. 5, the effects of anti-CD11b and anti-SR-B2 on the ingestion of α-syn and fibrillar α-syn (PFF) by N9 cells were observed. 10⁵ N9 cells were added to wells pre-coated with laminin, laminin and α-syn, or laminin and PFF α-syn for 24 hrs. Non-adherent cells were removed and either α-syn-coated or PFF α-syn-coated fl-spheres were added to the wells for 180 mins. The wells were then washed 4× with PBS and fluorescence was measured as arbitrary units using a cell plate reader. All values are normalized for cell number (using the CyQuant assay).

### Example 6: Effect of Anti-Receptor Antibodies on Phagocytosis and Down Modulation of N9 Receptors

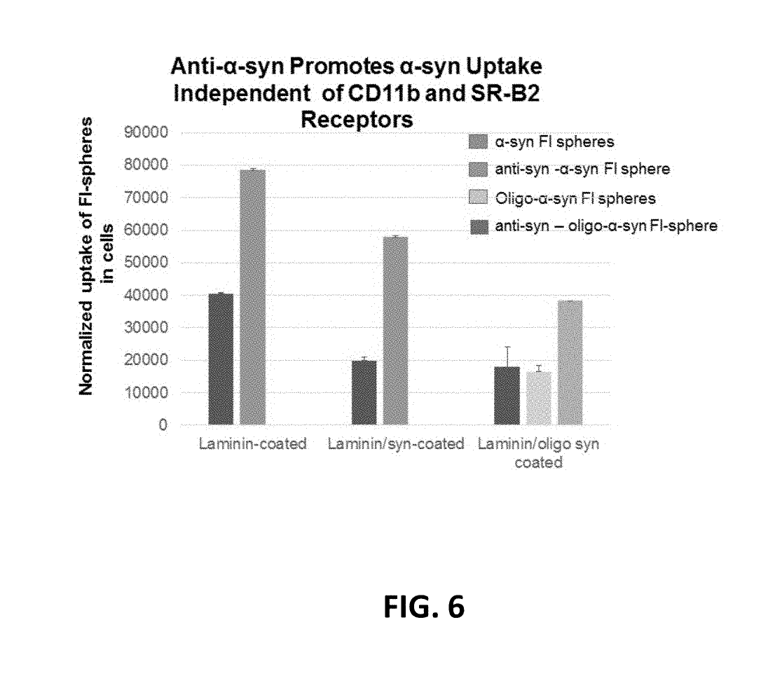

FIG. 6 shows the binding and uptake of Fl-spheres by cells in which the various receptors have been down-modulated (see FIG. 1). 100,000 N9 cells were added to wells pre-coated with laminin, laminin and α-syn, or laminin and oligomeric α-syn for 24 hours. Non-adherent cells were removed and incubated with either a) α-oligomerized α-syn coated, b) α-syn coated, c) anti-α-syn:α-syn coated, or d) anti-α-syn antibody:oligomerized α-syn coated Fl-spheres (100 beads/cell) to the wells for 60 minutes. Fl-sphere uptake was assessed as in Example 4.

### Example 7: Effects of Ant-CD11b and Anti-SR-B2 on the Ingestion of Monomeric Vs Oligomeric Proteins

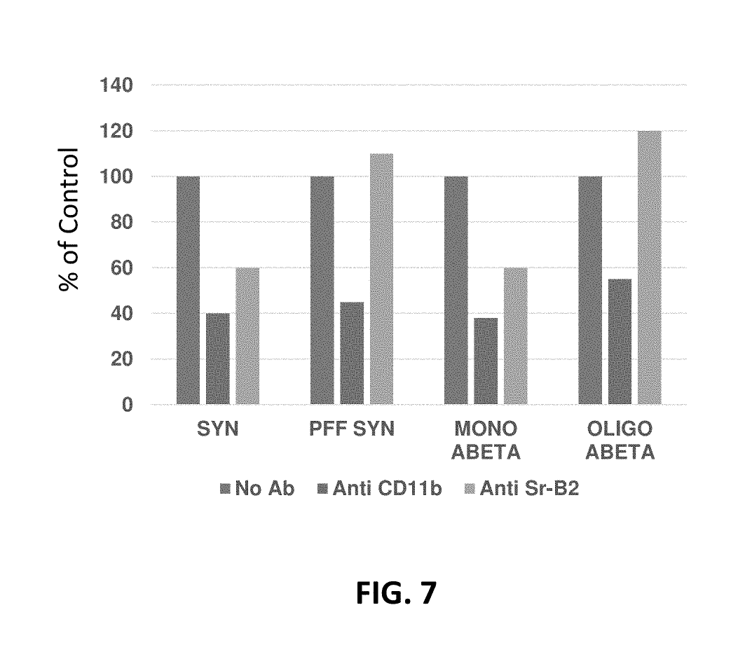

As seen in FIG. 7, the effects of ant-CD11b and anti-SR-B2 on the ingestion of monomeric vs oligomeric proteins were observed. Ingestion of monomeric α-syn-coated, monomeric Beta Amyloid (Abeta)-coated, PFF-coated and oligomeric Abeta-coated fl-spheres by microglial cells was measured. 10⁵ N9 cells were added for 24 hours to wells pre-coated with laminin. Non-adherent cells were removed and the indicated protein-coated fl-spheres were added to the wells for 180 minutes. The cells were then washed 5× with PBS and fluorescence was measured as arbitrary units using a cell plate reader. All values are normalized for cell number (using the CyQuant assay) and number of fl-spheres added.

### Example 8: Confocal Microscopy of N9 Cells Ingesting Monomeric α-Syn

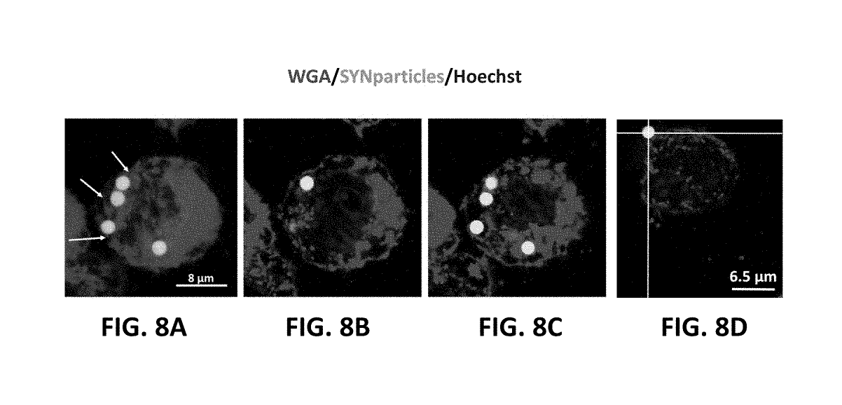

Cells were incubated with microspheres coated with monomeric α-synuclein conjugated with fluorochrome for 2 hours. After rigorous washing (×5), cells were stained with Wheat Germ Agglutinin (WGA) as a marker of plasma membrane and Hoechst for 15 min and examined with confocal microscope. Most of the microspheres were observed inside the cells (FIGS. 8A-8C). A few microspheres were attached to the plasma membrane, as shown in FIG. 8D.

### Example 9: Fibrillary Forms of α-Syn are Cleared Less Efficiently than Monomeric Forms

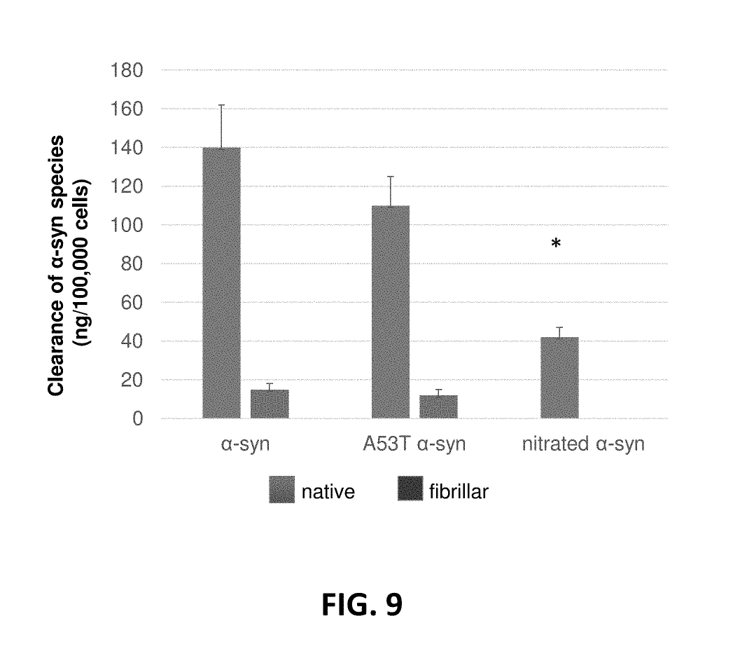

FIG. 9 shows clearance of monomeric or oligomeric α-syn. 100,000 N9 cells were incubated in lam-coated wells that were pre-coated with either 2 μg/well of Alexa labeled monomeric α-syn, PFF α-syn, monomeric mutant (A53T) α-syn, or fibrillar mutant (A53T) α-syn. At the end of 48 hours, cells were washed 3× with PBS and detached with 10 mM EDTA. Fluorescence associated with the cells was then measured using a cell plate reader.

### Example 10: Effect of SAW on α-Syn Uptake

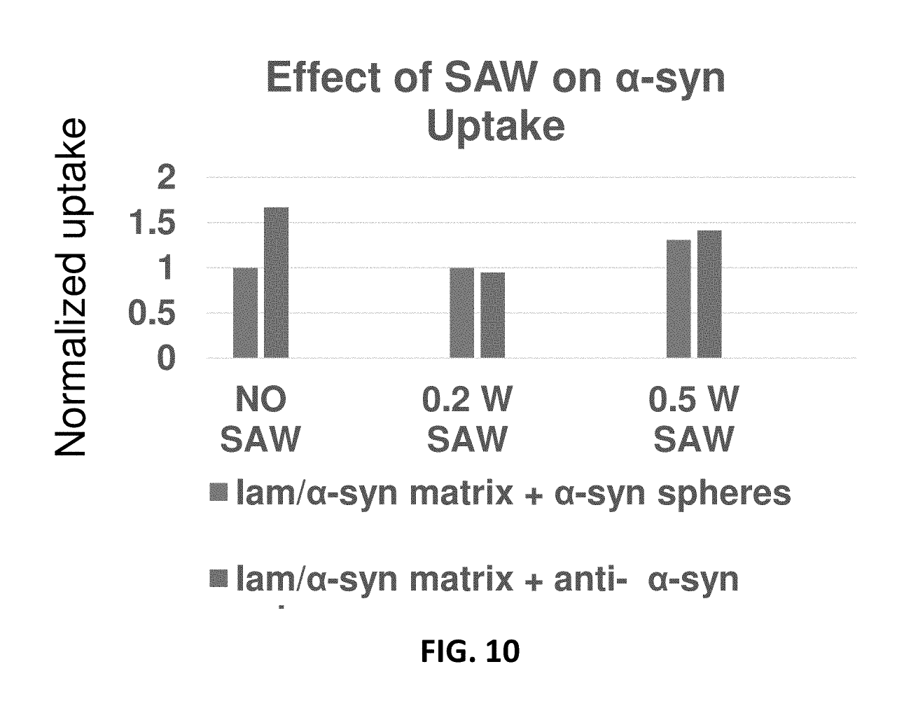

FIG. 10 shows the effect of SAW on α-syn uptake. Cells were incubated on a laminin/α-syn matrix with α-syn spheres or with anti-α-syn spheres. The cells were then subjected to 0.2 W SAW or 0.5 W SAW and the uptake of the spheres was measured.

### Example 11: Effects of SAW on TNFα and IL-6 Production by N9 Cells

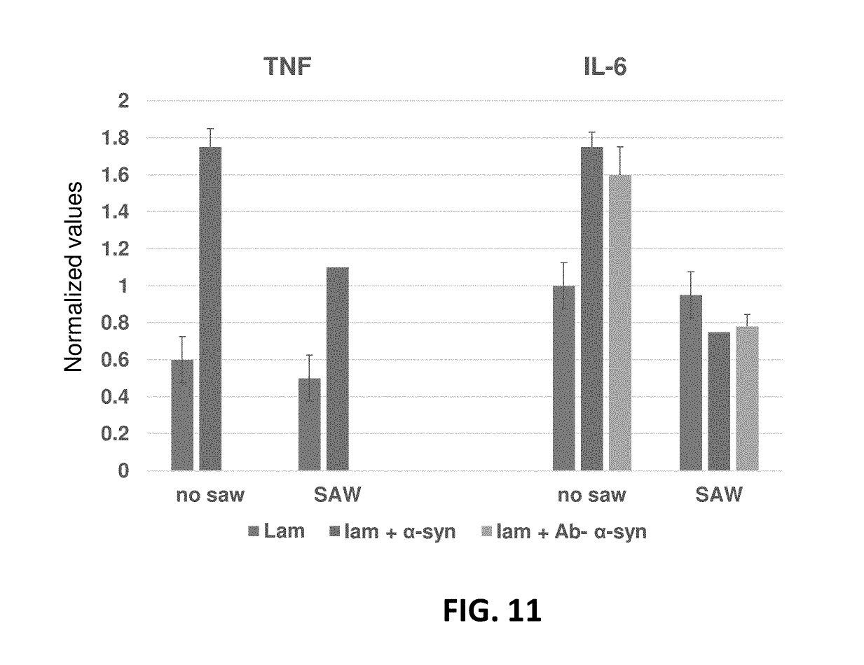

10⁵ N9 cells were incubated for 48 hours adherent to 3 μg/well of laminin, 3 μg/well of laminin+3 μg/well α-syn, or 3 μg/well of laminin+3 μg/well anti-Ab α-syn. The cells were then subjected to SAW or not subjected to SAW and cytokine production of TNF-α and IL-6 was measured by ELISA as described in the media, the results of which are shown in FIG. 11.

### Example 12: Effects of SAW on IL-6 Production

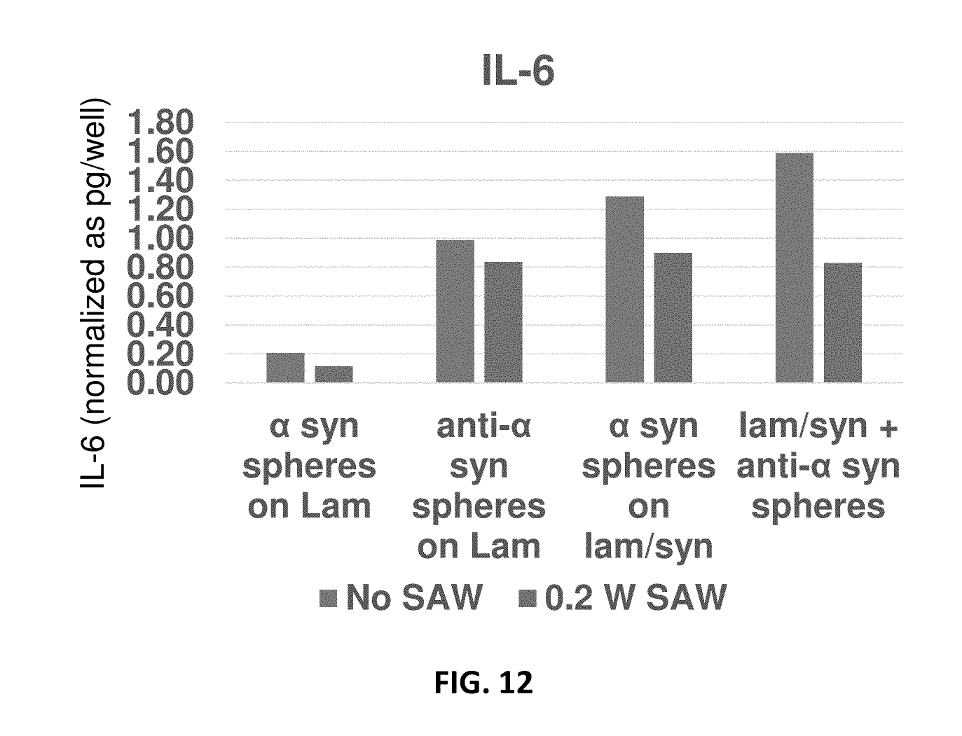

Cells were incubated on a laminin matrix with α-syn spheres, laminin with anti-Ab α-syn spheres, laminin and α-syn with α-syn spheres, or laminin and α-syn with anti-Ab α-syn spheres. The cells were then subjected to 0.2 W SAW or not subjected to SAW and cytokine production of IL-6 was measured by ELISA, the results of which are shown in FIG. 12.

### Example 13: Effects of SAW on N9 Ingestion of Protein-Coated Spheres

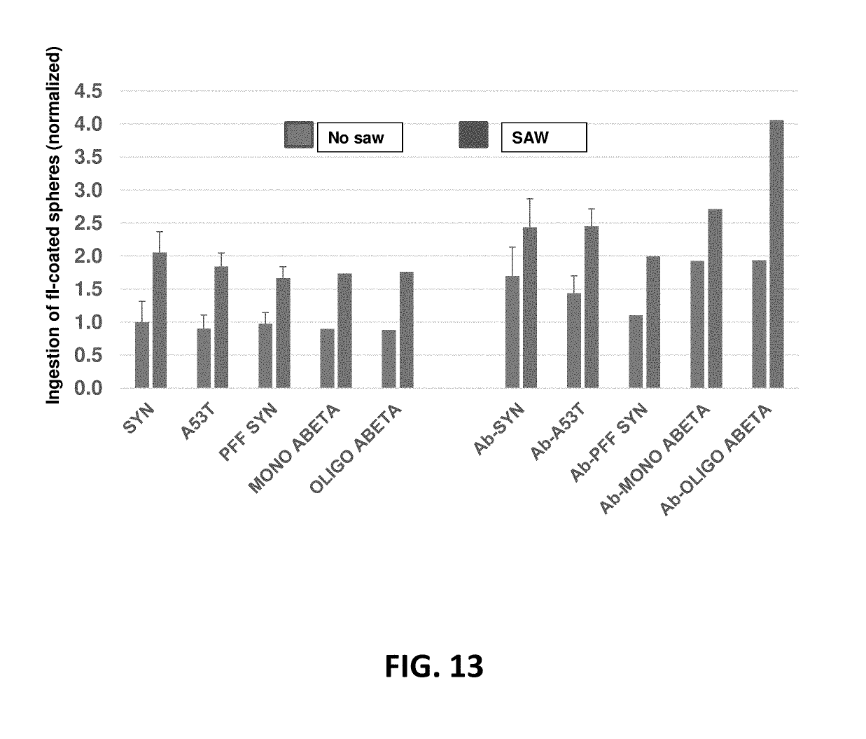

10⁵ N9 cells were added to wells pre-coated with laminin for 24 hours. α-syn-coated, A53T-coated, PFF-coated, monomeric Abeta-coated, oligomeric Abeta-coated, anti-α-syn-coated (Ab-SYN), anti-A53T-coated (Ab-A53T), anti-PFF-coated (Ab-PFF SYN), monomeric anti-Abeta-coated (Ab-MONO ABETA), oligomeric anti-Abeta-coated (Ab-OLIGO ABETA) fl-spheres were added to the wells for 180 mins. Cells were processed as described in Example 7. FIG. 13 shows that the application of SAW increased the ingestion of the unopsonized or opsonized protein-coated fl-spheres by microglial cells.

### Example 14: SAW and the BBB

SAW will be administered to the head of wild type and transgenic mice (modeled for either Parkinson's disease or Alzheimer's disease). Anti-synuclein antibody or anti-beta-amyloid antibody will be simultaneously injected to determine whether more antibodies cross the blood brain barrier to target the brain in the presence of SAW.

### Example 15: Measuring Inflammation Biomarkers from CSF

Cerebral spinal fluid (CSF) will be removed from untreated or SAW treated older transgenic mice (modeled for either Parkinson's disease or Alzheimer's disease) and ELISA technology will be used to measure biomarkers for inflammation. The expectation is that SAW will reduce the production of biomarkers of inflammation in the CSF of treated animals.

### Example 16: SAW with Therapeutic Antibodies

SAW will be administered to the head of mice in conjunction with therapeutic antibodies that attenuate behavioral deficits in either PD or Alzheimer's disease to assess its medical efficacy. The prediction is that in the presence of SAW, either fewer antibodies will be required or the antibodies will have a greater impact on resolving the behavior deficits.

Having described several embodiments, it will be recognized by those skilled in the art that various modifications, alternative constructions, and equivalents may be used without departing from the spirit of the invention. Additionally, a number of well-known processes and elements have not been described in order to avoid unnecessarily obscuring the present invention. Accordingly, the above description should not be taken as limiting the scope of the invention.

Those skilled in the art will appreciate that the presently disclosed embodiments teach by way of example and not by limitation. Therefore, the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

What is claimed is:

1. A method of reducing inflammation in an area of interest of a patient with a chronic condition, comprising:
applying surface acoustic waves to the area of interest, wherein the surface acoustic waves promote microglial cell ingestion and clearance of a target protein.

2. The method of claim 1, wherein the target protein is selected from the group consisting of α-synuclein, beta amyloid, and combinations thereof.

3. The method of claim 2, wherein the target protein is α-synuclein.

4. The method of claim 2, wherein the target protein is beta amyloid.

5. The method of claim 1, wherein the chronic condition is selected from the group consisting of Parkinson's disease, Alzheimer's disease, diabetes, food allergies, transplantation rejection, cancer, and erectile dysfunction.

6. The method of claim 5, wherein the chronic condition is Parkinson's disease.

7. The method of claim 5, wherein the chronic condition is Alzheimer's disease.

8. The method of claim 1, wherein the power of the surface acoustic waves is between about 0.2 W and about 0.5 W.

9. The method of claim 1 further comprising administering a target protein antibody selected from the group consisting of anti-α-synuclein antibody, anti-beta amyloid antibody, and combinations thereof.

10. The method of claim 1 further comprising administering a therapeutic antibody for the chronic condition.

11. The method of claim 1, wherein the application of surface acoustic waves decreases the secretion of TNF-α or IL-6.

12. A method for reducing inflammation in a patient with Parkinson's disease, comprising applying surface acoustic waves to the patient, wherein the surface acoustic waves promote ingestion and clearance of α-synuclein by microglial cells.

13. The method of claim 12, wherein the power of the surface acoustic waves is between about 0.2 W and about 0.5 W.

14. The method of claim 12 further comprising administering an anti-α-synuclein antibody.

15. The method of claim 12, wherein the application of surface acoustic waves decreases the secretion of TNF-α or IL-6.

16. The method of claim 12 further comprising administering a therapeutic antibody for Parkinson's disease.

17. A method for reducing inflammation in a patient with Alzheimer's disease, comprising applying surface acoustic waves to the patient, wherein the surface acoustic waves promote ingestion and clearance of beta amyloid by microglial cells.

18. The method of claim 17, wherein the power of the surface acoustic waves is between about 0.2 W and about 0.5 W.

19. The method of claim 16 further comprising administering an anti-beta amyloid antibody.

20. The method of claim 16, wherein the application of surface acoustic waves decreases the secretion of TNF-α or IL-6.
